(19) United States
(12) Patent Application Publication
(10) Pub. No.: US 2022/0036762 A1
(43) Pub. Date: Feb. 3, 2022

(54) SYSTEM FOR EXERCISE PROGRAM MANAGEMENT

(71) Applicant: Cody Brown, Bremerton, WA (US)

(72) Inventor: Cody Brown, Bremerton, WA (US)

(21) Appl. No.: 16/983,234

(22) Filed: Aug. 3, 2020

(51) Int. Cl. G09B 19/00; G16H 20/30; G16H 20/60; G06Q 10/10; G06Q 20/08; G16H 80/00; G06Q 50/00; G06F 16/9535; G09B 5/06

(52) U.S. Cl. CPC G09B 19/0092; G16H 20/30; G16H 20/60; G06Q 10/1093; G09B 5/065; G16H 80/00; G06Q 50/01; G06F 16/9535; G09B 19/003; G06Q 20/085

(57) ABSTRACT

A computerized system for health management is disclosed, comprising a database configured to store a plurality of physical exercise and nutrition data input by a user via a computing device. A calendar module is in operable communication with a scheduling module to permit the user to schedule exercise information and nutritional information. A communications module permits communication between a client and a provider. A media module is in operable communication with a camera to receive video information and associate the video information with one or more exercises. The video information is provided on an exercise interface to permit the user to engage with the video information.

### TECHNICAL FIELD

The embodiments generally relate to computerized systems for health management and, more specifically, relate to computerized systems for managing personal fitness and physical training in a network environment.

### BACKGROUND

Physical fitness is an essential part of maintaining a healthy lifestyle. Physical fitness refers to proper nutrition, physical exercise, and sufficient rest. Each aspect of physical fitness has various protocols for best practices related to specific goals. The physical fitness and personal training industries generate over $100 billion U.S. dollars in revenue each year.

Each year, millions of people struggle with fitness goals due to lack of motivation, lack of proper guidance, and inadequate knowledge. Aggregating fitness data allows individuals to monitor fitness progress, which in-turn helps individuals stay motivated over long periods of time. However, tracking fitness data can be complex, leading to non-compliance.

Improvements in mobile technologies have allowed for the advancement of monitoring health data. Users can now easily monitor physical well-being, fitness goals, sleep statistics, and nutritional information which can be stored in a database and provided on a user interface for analysis. While tracking data has been made easy, many individuals prefer to receive training programs from a professional personal trainer. This removes the need to research exercise and nutritional advice, which the personal trainer may provide as a service.

Personal trainers are individuals certified to have a varying degree of fitness knowledge, including exercise prescription and instruction. Some personal trainers may also provide nutritional advice tailored to their clients' goals, such as weight loss, mass gain, or sport-specific meal plans. Many physical trainers operate at a gym and engage with clients in-person; however, virtual training and consultations have become increasingly popular.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The embodiments provided herein relate to a computerized system for health management, comprising a database configured to store a plurality of physical exercise and nutrition data input by a user via a computing device. A calendar module is in operable communication with a scheduling module to permit the user to schedule exercise information and nutritional information. A communications module permits communication between a client and a provider. A media module is in operable communication with a camera to receive video information and associate the video information with one or more exercises. The video information is provided on an exercise interface to permit the user to engage with the video information.

The embodiments further provide a computerized system for the aggregation and management of physical fitness and nutritional information. The system allows for users to interact with physical fitness and nutrition information as well as with other users over a network. Clients may seek providers of nutrition, exercise, or similar health and well-being information. The clients may search a listing of providers to view user profiles containing information related to the user. For example, a client may search for a personal trainer with expertise in weight loss by viewing profiles of providers. The users may then engage with one another by communicating over the network, engage with virtual training, or engage with physical exercise, nutrition, or general health information.

In one aspect, a payment processor processes payments transmitted from the client to the provider.

In one aspect, a permissions module determines exercise information configured behind a paywall.

In one aspect a user profile interface provides a user profile provided by the user.

In one aspect, a search engine permits the user to search user profiles stored in the database.

In one aspect, the exercise interface comprises imagery of an exercise and an instruction for performing the exercise.

In one aspect, the exercise interface is configured to categorically provide exercise by at least one of the following: body region, muscle group, exercise goal, and experience level.

In one aspect, a nutrition interface is in operable communication with the calendar interface to permit the user to input nutrition information.

In one aspect, the nutrition information is transmitted to the provider to permit the provider to input nutrition suggestions.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of the network infrastructure, according to some embodiments;

FIG. 2 illustrates a screenshot of the user profile interface, according to some embodiments;

FIG. 3 illustrates a screenshot of the user profile interface, according to some embodiments;

FIG. 4 illustrates a screenshot of the exercise interface, according to some embodiments;

FIG. 5 illustrates a screenshot of the nutrition interface, according to some embodiments;

FIG. 6 illustrates a screenshot of the exercise detail interface, according to some embodiments;

FIG. 7 illustrates a screenshot of the supplement interface, according to some embodiments;

FIG. 8 illustrates a screenshot of the user settings interface, according to some embodiments;

FIG. 9 illustrates a schematic of the nutrition and physical exercise communications system, according to some embodiments; and

FIG. 10 illustrates a block diagram of the server engine and modules, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood therefrom.

Before describing in detail exemplary embodiments, it is noted that the embodiments reside primarily in combinations of components and procedures related to the system. Accordingly, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

As used herein, the term “user” refers to individuals and organizations using the system to interact with health information, physical fitness information, nutritional information, and communications over a network. Users may include personal trainers, nutritionists, or other health and physical fitness experts who provide information for consumption by clients or other users of the system. Users may also include persons tracking physical exercise data, engaging with personal trainers, nutritionists, and health experts, and otherwise engaging with the system. At least some users may be defined as clients engaging with the services of a physical trainer, nutritionist, or other health and wellness professional.

As used herein, the term “provider” refers to users (individuals or organizations) utilizing the system to provide a service or information for consumption by clients. The term “client” may be utilized herein to refer to users consuming information provided by the providers.

In general, the system described herein relates to a computerized system for the aggregation and management of physical fitness and nutritional information. The system allows for users to interact with physical fitness and nutrition information as well as with other users over a network. Clients may seek providers of nutrition, exercise, or similar health and well-being information provided by the providers. The clients may search a listing of providers to view user profiles containing information related to the user. For example, a client may search for a personal trainer with expertise in weight loss by viewing profiles of providers. The users may then engage with one another by communicating over the network, engage with virtual training, or engage with physical exercise, nutrition, or general health information.

The system provided herein allows for client and provider communications to be transmitted via network communications to allow remote consultations, training, and similar interactions. The system allows for providers to advertise, promote, and provide services on a platform used by their clients. Providers will be able to create and transmit information related to individualized programs including physical fitness programs, workout regimes, nutritional plans, and recommendations for supplements.

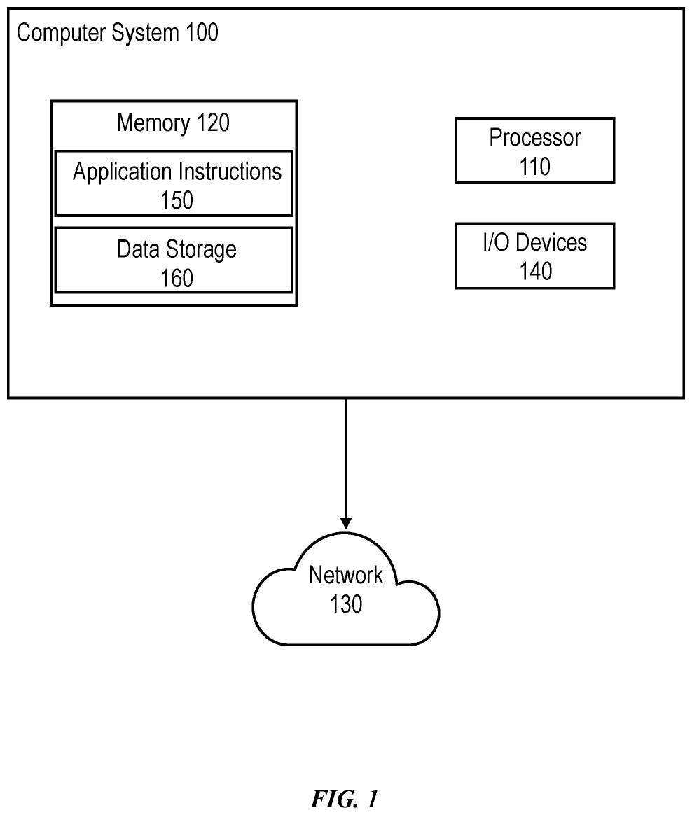

FIG. 1 illustrates a computer system 100, which may be utilized to execute the processes described herein. The computer system 100 is comprised of a standalone computer or mobile computing device, a mainframe computer system, a workstation, a network computer, a desktop computer, a laptop, or the like. The computer system 100 includes one or more processors 110 coupled to a memory 120 via an input/output (I/O) interface. Computer system 100 may further include a network interface to communicate with the network 130. One or more input/output (I/O) devices 140, such as video device(s) (e.g., a camera), audio device(s), and display(s) are in operable communication with the computer system 100. In some embodiments, similar I/O devices 140 may be separate from computer system 100 and may interact with one or more nodes of the computer system 100 through a wired or wireless connection, such as over a network interface.

Processors 110 suitable for the execution of a computer program include both general and special purpose microprocessors and any one or more processors of any digital computing device. The processor 110 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 100 and other devices attached to a network 130, such as other computer systems, or between nodes of the computer system 100. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

The memory 120 may include application instructions 150, configured to implement certain embodiments described herein, and a database 160, comprising various data accessible by the application instructions 150. In one embodiment, the application instructions 150 may include software elements corresponding to one or more of the various embodiments described herein. For example, application instructions 150 may be implemented in various embodiments using any desired programming language, scripting language, or combination of programming languages and/or scripting languages (e.g., C, C++, C#, JAVA®, JAVASCRIPT®, PERL®, etc.).

In some embodiments, the memory 120 is configured to store information relates to fitness, nutrition, sleep/rest, and general health and wellness. Information may be stored in a text form, audio format, and/or video format. Further, the memory 120 may include user settings, profile settings, user preference, and user permissions. The user permissions may be provided by an administrator.

The steps and actions of the computer system 100 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

To interact with the system, users register with the system by providing user credentials as is known in the arts. User credentials may include login information, social media information, or other identifying credentials which may associate the user with external social media systems. The user may create a profile containing personal information, anthropometric data, imagery, goals, achievements, and other useful information.

In some embodiments, providers may create regimes, programs, or similar content related to specific exercises, workouts, nutrition, supplements, and other useful information, which is provided on their profile with which the client may engage. Some information may be configured to provide a paywall, such that the client must provide payment for at least a portion of the information provided by the provider. One skilled in the arts will readily understand that providers may select various payment schemes, provide information without requiring payment, offer trial periods, and the like.

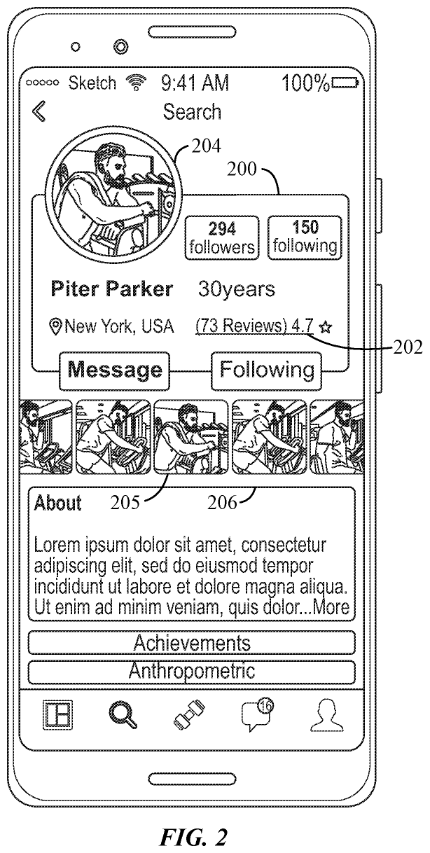

FIG. 2 illustrates the user profile interface 200 having a plurality of user information related to the user. For example, the user profile interface 200 may include networking information such as the number of followers, number of users the user is following, user location, user experience, and user identifying information. A reviews tab 202 allows for users to interact with reviews submitted by other users or input a review related to the user. For example, the user profile 204 may be related to a provider who is a personal trainer. Clients may select the reviews tab 202 to read reviews from previous or current clients, as well as review the services provided by the provider. The reviews are stored in the database. Imagery 205 may be provided on the user profile interface 200 which may be selected by the user viewing the user profile 204. An information section 206 provides information related to the user to whom the profile relates.

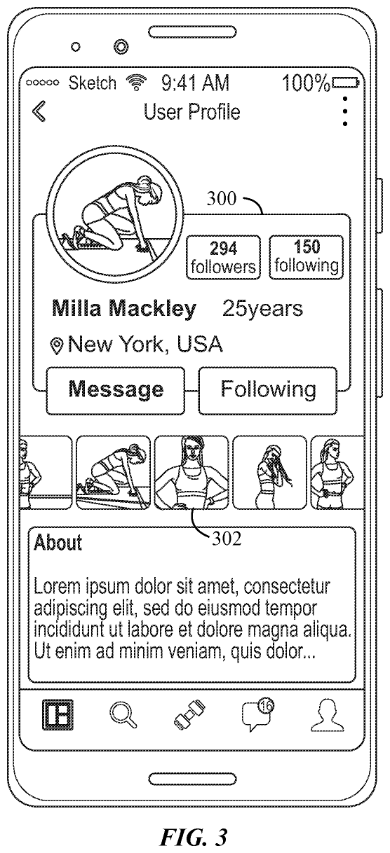

FIG. 3 illustrates a client profile interface 300 whereon client information is provided. The client profile interface 300 may include client imagery 302 to allow the client to store imagery related to their physical fitness goals. Further, the client may utilize the client imagery to store exercise information or store imagery provided by other users.

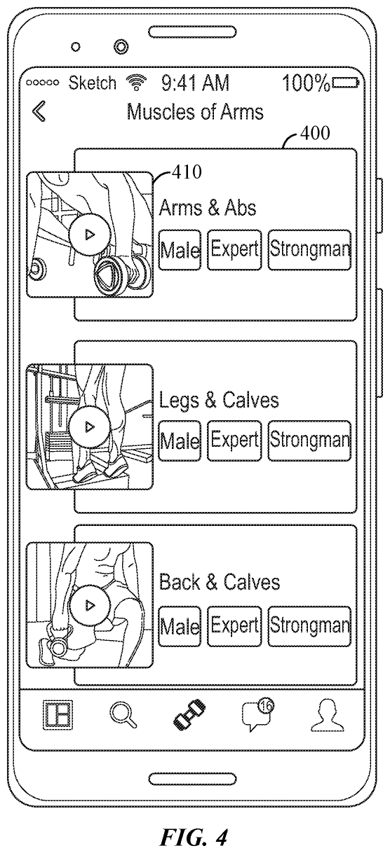

FIG. 4 illustrates the exercise interface 400 having a plurality of information related to exercises. The exercise information may be provided by the provider and may include exercise categories, specific exercises, and the relation thereof to the user's goals. The exercise interface 400 allows the user to search various exercises based on body region, muscle group, or exercise goals. For example, the user may select “arms and abs” and select strongman to view exercises which train the arms and abs for strongman-specific training. A video link 410 permits the user to select a video which corresponds to the exercise regime they have selected. For example, selecting a video link 410 for an arms and abs workout for a strongman will transmit the user to a video interface showing a video, imagery, or likewise information related to the exercise.

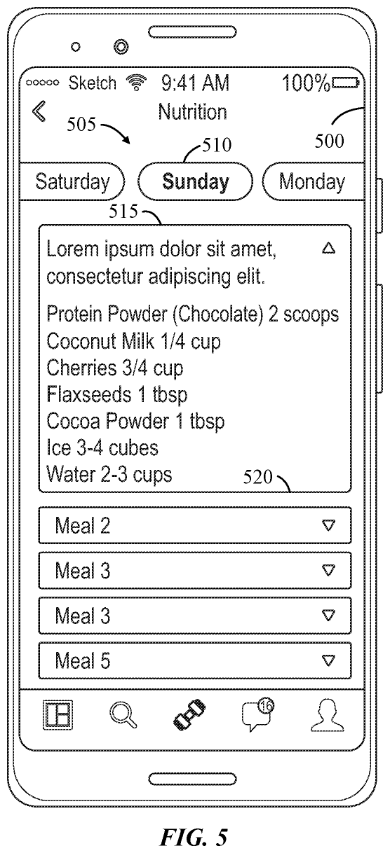

FIG. 5 illustrates a nutrition interface 500 allowing the user to input nutrition information. The nutrition information may be segmented in a calendar 505, allowing the user to separate nutrition information by the day of the week, the week, the month, or other period of time. For example, the user selects a time period 510 (e.g., Sunday) and inputs nutrition information 515 into a nutrition input field 520, such as listing food items they have eaten on a particular day. Alternatively, as illustrated, the user may input recipes or nutritional information (e.g., amounts of calories, fats, protein, etc.).

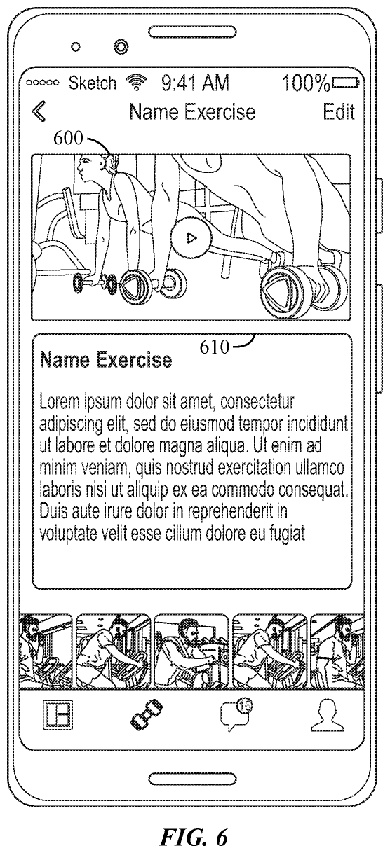

FIG. 6 illustrates a screenshot of the exercise detail interface 600 which provides information for a specific exercise. The exercise information may include one or more videos or imagery detailing the exercise as well as instructions 610 for performing the exercise. For example, a provider may provide information related to a deadlift exercise with a video demonstrating the proper techniques for performing a deadlift. The provider may also input written instructions to supplement the video instructions. Exercise information may also provide details related to the exercise benefits, etc.

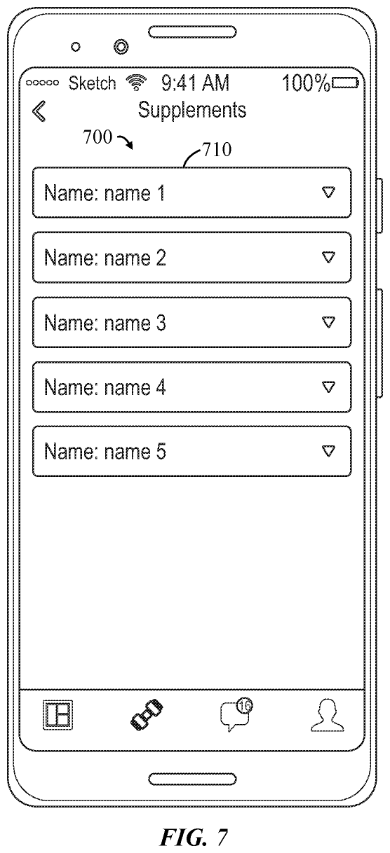

FIG. 7 illustrates the supplements interface 700 whereon the user may store supplement information 710 including the supplement name, supplement recommended use, supplement nutritional information, supplement directions, and the like. The supplement interface 700 may be used by the provider to indicate supplements the client is instructed to consume, or for the client to indicate supplements they are consuming.

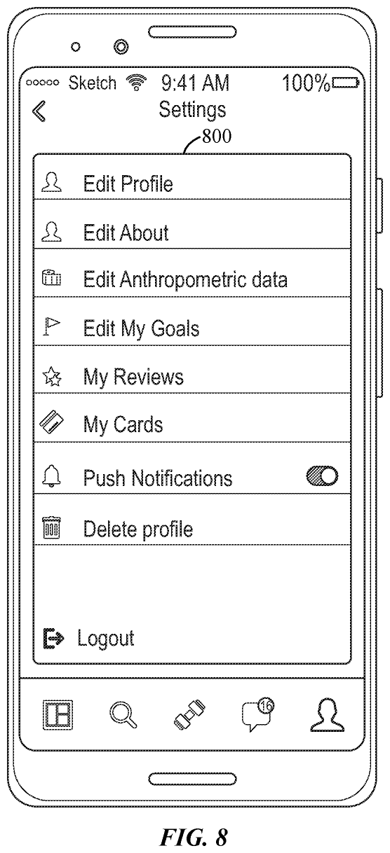

FIG. 8 illustrates the settings interface 800 to permit the user to input user settings. User settings may include profile settings, user information, anthropometric data, goals, reviews, payment options, notification settings, and similar settings known in the arts. A payment processor permits the user to send and/or receive payments to users of the system described hereinabove. For example, a client may transmit payment to a provider for engaging with services provided by the provider.

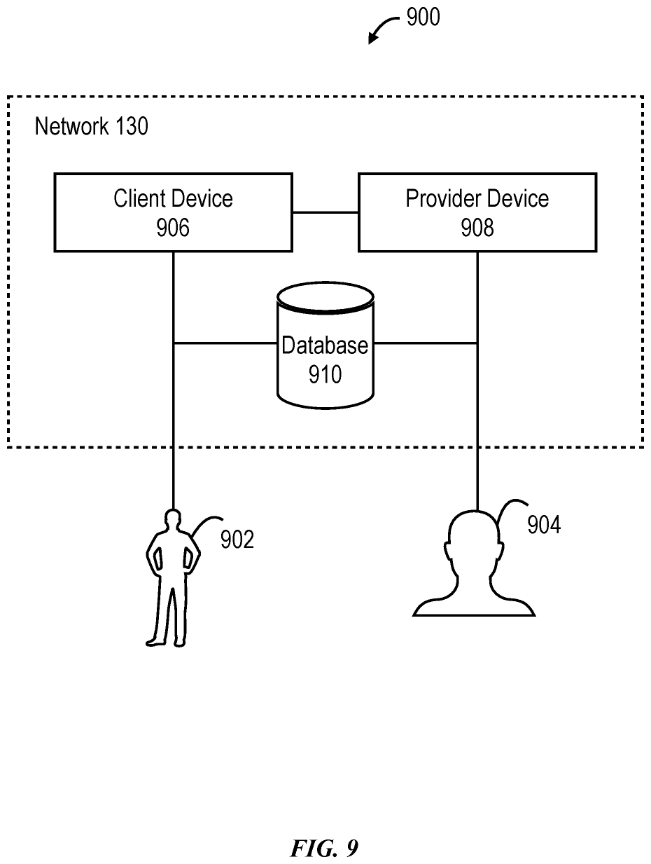

FIG. 9 illustrates a schematic of the system 900 including a client 902 and provider 904 each in operable communication with a client device 906 and provider device 908 respectively. The client 902 utilizes their client device 906 to interact with the system as described herein, including interacting with information provided by the system and users thereof. The client 902 may communicate with the provider 904 via the network 130 to receive exercise, nutrition, and general health and wellbeing information. A database 910 stores information received from the client device 906 and provider device 908.

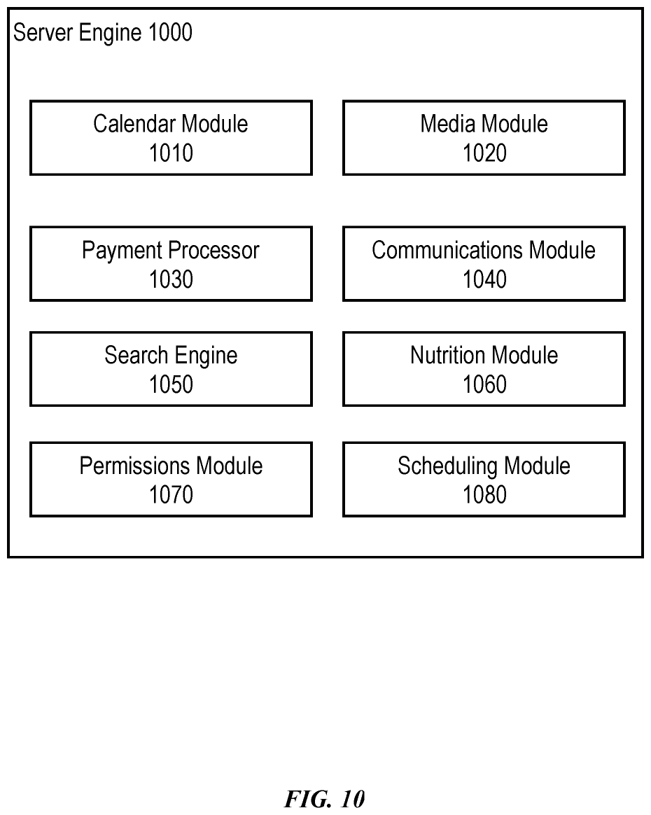

FIG. 10 illustrates a block diagram of the server engine 1000 and modules. A calendar module 1010 is in operable communication with a scheduling module 1080 to provide calendar functions, allowing the user to schedule exercise routines, schedule nutrition plans, and schedule communications between users. For example, a client may utilize the calendar module 1010 to input daily nutritional intake and exercise routines. The scheduling module 1080 allows the client to view the schedule of a provider and reserve a time period wherein they may contact the provider or engage in a virtual interaction with the provider. A provider may use the scheduling module 1080 to input availabilities and receive requests from interested clients. The provider may utilize the calendar module 1010 to input exercise regime schedules, nutrition information, and the like to be transmitted to the client. A media module 1020 permits the user to input media (e.g., instructional videos, audio, or imagery). The media module 1020 may receive video, audio, or imagery information from the camera of the computing device. In one example, the media module 1020 allows the user to correspond media information with an exercise that is displayed on the exercise interface described hereinabove. A payment processor 1030 processes payment information between users of the system as is known in the arts. A communications module 1040 provides communication functions between the client and the provider. A search engine 1050 allows the user to search information provided by the system, including searching listings of clients, listings of providers, listings of exercises, listings of nutritional information, and the like. A nutrition module 1060 provides nutritional information and calculates nutritional totals related to the user's diet. For example, the nutrition module 1060 may associate a nutritional fact values with a food item to calculate a total value for the diet of the user. A permissions module 1070 receives permissions from an administrative user to determine if a user has permission to view particular information. For example, the permissions module 1070 determines if the user has paid for information provided by a provider and allows the client to view the information configured behind a paywall.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A computerized system for health management, comprising:
a database configured to store a plurality of physical exercise and nutrition data input by a user via a computing device;
a calendar module in operable communication with a scheduling module to permit the user to schedule exercise information and nutritional information;
a communications module to permit communication between a client and a provider;
a media module in operable communication with a camera to receive video information and associate the video information with one or more exercises, wherein the video information is provided on an exercise interface.

2. The system of claim 1, further comprising a payment processor to process payments transmitted from the client to the provider.

3. The system of claim 2, wherein a permissions module determines exercise information configured behind a paywall.

4. The system of claim 1, wherein a user profile interface provides a user profile provided by the user.

5. The system of claim 4, wherein a search engine permits the user to search user profiles stored in the database.

6. The system of claim 1, wherein the exercise interface comprises imagery of an exercise and an instruction for performing the exercise.

7. The system of claim 1, wherein the exercise interface is configured to categorically provide exercise by at least one of the following: body region, muscle group, exercise goal, and experience level.

8. The system of claim 1, further comprising a nutrition interface in operable communication with the calendar interface, wherein the nutrition interface permits the user to input nutrition information.

9. The system of claim 8, wherein the nutrition information is transmitted to the provider to permit the provider to input nutrition suggestions.

10. A computerized system for health management, comprising:
a database configured to store a plurality of physical exercise and nutrition data input by a user via a computing device;
a calendar module in operable communication with a scheduling module to permit the user to schedule exercise information and nutritional information;
a communications module to permit communication between a client and a provider;
a media module in operable communication with a camera to receive video information and associate the video information with one or more exercises, wherein the video information is provided on an exercise interface;
a nutrition interface to permit the user to input nutrition information and transmit the nutrition information between the client and the provider; and
a nutrition module in operable communication with the nutrition interface, wherein the nutrition module aggregates nutrition information and calculates nutrition totals to correspond the nutrition total with a period of time.

11. The system of claim 10, further comprising a payment processor to process payments transmitted from the client to the provider.

12. The system of claim 11, wherein a permissions module determines exercise information configured behind a paywall.

13. The system of claim 12, wherein a user profile interface provides a user profile provided by the user.

14. The system of claim 13, wherein a search engine permits the user to search user profiles stored in the database.

15. The system of claim 14, wherein the exercise interface comprises imagery of an exercise and an instruction for performing the exercise.

16. The system of claim 15, wherein the exercise interface is configured to categorically provide exercise by at least one of the following: body region, muscle group, exercise goal, and experience level.

17. The system of claim 16, wherein the nutrition interface in operable communication with the calendar interface, wherein the nutrition interface permits the user to input nutrition information corresponding to a period of time.

18. The system of claim 17, wherein the nutrition information is transmitted to the provider to permit the provider to input nutrition suggestions.

19. The system of claim 18, further comprising a supplement interface to receive supplement information input by the user.

20. A computerized system for health management, comprising:
a database configured to store a plurality of physical exercise and nutrition data input by a user via a computing device;
a calendar module in operable communication with a scheduling module to permit the user to schedule exercise information and nutritional information;
a communications module to permit communication between a client and a provider;
a media module in operable communication with a camera to receive video information and associate the video information with one or more exercises, wherein the video information is provided on an exercise interface;
a nutrition interface to permit the user to input nutrition information and transmit the nutrition information between the client and the provider;
a nutrition module in operable communication with the nutrition interface, wherein the nutrition module aggregates nutrition information and calculates nutrition totals to correspond the nutrition total with a period of time via the calendar module;
a supplement interface to receive supplement information from the user and schedule, via the calendar module, the supplement information to schedule the consumption of supplements.
